(19) United States
(12) Patent Application Publication
(10) Pub. No.: US 2022/0000880 A1
(43) Pub. Date: Jan. 6, 2022

(54) METHOD AND COMPOSITION EMBODIMENTS FOR TREATING ACUTE MYELOID LEUKEMIA

(71) Applicant: Rigel Pharmaceuticals, Inc., South San Francisco, CA (US)

(72) Inventors: Sandra Tong, Los Altos, CA (US); Vadim Markovtsov, San Mateo, CA (US); Ann Lowe, Austin, TX (US); Anne-Marie Duliege, Palo Alto, CA (US)

(73) Assignee: Rigel Pharmaceuticals, Inc., South San Francisco, CA (US)

(21) Appl. No.: 17/290,741

(22) PCT Filed: Oct. 31, 2019
(86) PCT No.: PCT/US2019/059268

§ 371 Date: Apr. 30, 2021

Related U.S. Application Data
(60) Provisional application No. 62/753,979, filed on Nov. 1, 2018.

(51) Int. Cl. A61K 31/5383; A61K 31/675; A61K 31/5377; A61K 31/7076; A61K 31/704; A61K 31/7068; A61K 31/635; A61K 31/4745; A61K 31/553; A61K 38/20; A61K 39/395; A61K 31/417; A61K 31/53; A61P 35/00

(52) U.S. Cl. CPC A61K 31/5383; A61K 31/675; A61K 31/5377; A61K 31/7076; A61K 31/704; A61K 31/7068; A61P 35/00; A61K 31/4745; A61K 31/553; A61K 38/2013; A61K 39/3955; A61K 31/417; A61K 31/53; A61K 31/635

(57) ABSTRACT

Disclosed herein are embodiments of a method and pharmaceutical composition for treating acute myeloid leukemia (AML). In particular, the method embodiments comprise treating AML with 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, alone or in combination with one or more therapeutic agents that themselves are effective for treating AML. Also disclosed are embodiments of a pharmaceutical composition comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, either as the sole therapeutic agent or in combination with one or more therapeutic agents effective for treating AML.

### CROSS REFERENCE TO RELATED APPLICATION

This application claims the benefit of the earlier filing date of U.S. Provisional Patent Application No. 62/753,979, filed on Nov. 1, 2018, the entirety of which is incorporated herein by reference.

### BACKGROUND

Acute myeloid leukemia (AML), also known as acute myelogenous leukemia, acute myeloblastic leukemia, acute granulocytic leukemia, or acute nonlymphocytic leukemia, is a fast-growing form of cancer of the blood and bone marrow. It is the most common type of acute leukemia. It occurs when the bone marrow begins to make blasts, cells that have not yet completely matured. These blasts normally develop into white blood cells; however, in AML, these cells do not develop and are unable to ward off infections. Each year, 20,000 new cases are diagnosed and about 10,000 deaths occur. The median survival in relapsed/refractory AML is 5 months; 1 year survival is only 20%. There exists a need in the art for new therapeutic agents and pharmaceutical compositions for treating acute myeloid leukemia.

### FIELD

The present disclosure concerns pharmaceutical composition embodiments and method embodiments of making and using such pharmaceutical compositions to treat acute myeloid leukemia (AML).

### SUMMARY

Disclosed herein are embodiments of a method comprising administering a therapeutically effective amount of (i) 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, and (ii) a second therapeutic agent to a subject, wherein the subject has, or is at risk of developing, AML. In some embodiments, the method can comprise administering the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the prodrug thereof, in combination with (e.g., simultaneously or sequentially) one or more (e.g., two, or two or more) therapeutic agents or treatment regimens for treating AIL in a subject in need thereof.

Also disclosed are embodiments of a pharmaceutical composition comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, in an amount effective to treat AML. In some embodiments, the pharmaceutical composition further comprises a second therapeutic agent present in an amount effective to treat AML.

Also disclosed are embodiments of a kit comprising (i) 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a phosphate-containing prodrug thereof; and (ii) instructions for treating AML with the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof.

### DETAILED DESCRIPTION

### I. Overview of Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

“Phosphate” refers to the group —O—P(O)(OR′)₂, where each —OR′ independently is —OH; —O-aliphatic, such as —O-alkyl or —O-cycloalkyl; —O-aromatic, including both —O-aryl and —O— heteroaryl; —O-aralkyl; or —OR′ is —O⁻M⁺, where M is a counter ion with a single positive charge. Each M⁺ may be an alkali ion, such as K⁺, Na⁺, Li⁺; an ammonium ion, such as ⁺N(R″)₄ where R″ is H, aliphatic, heteroaliphatic, or aromatic (including both aryl and heteroaryl); or an alkaline earth ion, such as [Ca²⁺]_{0.5}, [Mg²⁺]_{0.5}, or [Ba²⁺]_{0.5}. In some embodiments, this term is used when specifying a particular class of prodrugs and/or progroups, such as “phosphate-containing” prodrugs, which can comprise a phosphate-containing progroup. In some embodiments, a representative “phosphate-containing” progroup can be a phosphonooxyalkyl group, which refers to the group -alkyl-phosphate, such as, for example, —CH₂OP(O)(OH)₂, or a salt thereof, such as —CH₂OP(O)(O⁻Na⁺)₂. In some embodiments, another representative “phosphate-containing” progroup can comprise a (((dialkoxyphosphoryl)oxy)alkyl) group, which refers to the dialkyl ester of a phosphonooxyalkyl group, such as, for example, —CH₂OP(O)(O-tert-butyl)₂.

“Patient” or “Subject” may refer generally to any living being, but more typically refers to mammals and other animals, particularly humans. Thus disclosed methods are applicable to both human therapy and veterinary applications.

“Pharmaceutically acceptable excipient” refers to a substance, other than the active ingredient, that is included in a formulation of the active ingredient. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Excipients can include, but are not limited to, antiadherents, binders, coatings, enteric coatings, disintegrants, flavorings, sweeteners, colorants, lubricants, glidants, sorbents, preservatives, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethyleneglycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin.

An “adjuvant” is a component that modifies the effect of other agents, typically the active ingredient. Adjuvants are often pharmacological and/or immunological agents. An adjuvant may modify the effect of an active ingredient by increasing an immune response. An adjuvant may also act as a stabilizing agent for a formulation. Exemplary adjuvants include, but are not limited to, aluminum hydroxide, alum, aluminum phosphate, killed bacteria, squalene, detergents, cytokines, paraffin oil, and combination adjuvants, such as Freund's complete adjuvant or Freund's incomplete adjuvant.

“Pharmaceutically acceptable carrier” refers to an excipient that is a carrier or vehicle, such as a suspension aid, solubilizing aid, or aerosolization aid. Remington: The Science and Practice of Pharmacy, The University of the Sciences in Philadelphia, Editor, Lippincott, Williams, & Wilkins, Philadelphia, Pa., 21^{st} Edition (2005), incorporated herein by reference, describes exemplary compositions and formulations suitable for pharmaceutical delivery of one or more therapeutic compositions and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

“Pharmaceutically acceptable salt” refers to pharmaceutically acceptable salts of a compound that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed compounds form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as amino acids, formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, xinafoic acid and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, tris(hydroxymethyl)aminomethane (Tris), ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, tris(hydroxymethyl)aminomethane (Tris), ethanolamine, trimethylamine, dicyclohexylamine, choline, and caffeine. (See, for example, S. M. Berge, et al., “Pharmaceutical Salts,” J. Pharm. Sci., 1977; 66:1-19, the relevant portion of which is incorporated herein by reference.) In particular disclosed embodiments, the compounds may be a formate, trifluoroactate, hydrochloride or sodium salt.

“Effective amount” with respect to a compound or pharmaceutical composition refers to an amount of the compound or pharmaceutical composition sufficient to achieve a particular desired result, such as to inhibit a protein or enzyme. In particular embodiments, an “effective amount” is an amount sufficient to inhibit Syk, FLT3, IDH1, or other kinase or compound that may be involved in AML; to elicit a desired biological or medical response in a tissue, system, subject or patient; to treat a specified disorder or disease; to ameliorate or eradicate one or more of its symptoms; and/or to prevent the occurrence of the disease or disorder. The amount of a compound which constitutes an “effective amount” may vary depending on the compound, the desired result, the disease state and its severity, the size, age, and gender of the patient to be treated and the like, as will be understood by a person of ordinary skill in the art, particularly with the benefit of the present disclosure.

“Prodrug” refers to compounds that are transformed in vivo to yield a biologically active compound, or a compound more biologically active than the parent compound. In vivo transformation may occur, for example, by hydrolysis or enzymatic conversion. Common examples of prodrugs include, but are not limited to, ester and amide forms of a compound having an active form bearing a carboxylic acid moiety. Examples of pharmaceutically acceptable esters of the compounds of the present disclosure include, but are not limited to, esters of phosphate groups and carboxylic acids, such as aliphatic esters, particularly alkyl esters (for example C₁₋₆alkyl esters). Other prodrug moieties include phosphate esters, such as —(CR^{d}R^{d})_{y}—O—P(O)(OH)(OH), or a salt thereof, wherein y is an integer ranging from 1 to 3, typically 1 or 2; and each R^{d} is, independently of the others, selected from hydrogen, substituted or unsubstituted lower alkyl, substituted or unsubstituted phenyl, substituted or unsubstituted methyl and substituted or unsubstituted benzyl. Acceptable esters also include cycloalkyl esters and arylalkyl esters, such as, but not limited to benzyl. Examples of pharmaceutically acceptable amides of the compounds of this invention include, but are not limited to, primary amides, and secondary and tertiary alkyl amides (for example with between about one and about six carbons). Amides and esters of disclosed exemplary embodiments of compounds according to the present invention can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Suboptimal Dose” is a dose typically used in a single administration to a patient in monotherapy or in standard of care combination therapies.

“Syk Kinase” refers to the well-known 72 kDa non-receptor (cytoplasmic) spleen protein tyrosine kinase expressed in B-cells and other hematopoetic cells. Syk kinase includes two consensus Src-homology 2 (SH2) domains in tandem that bind to phosphorylated immunoreceptor tyrosine-based activation motifs (“ITAMs”), a “linker” domain and a catalytic domain (for a review of the structure and function of Syk kinase see Sada et al., 2001, J. Biochem. (Tokyo) 130:177-186); see also Turner et al., 2000, Immunology Today 21:148-154). Syk kinase has been extensively studied as an effector of B-cell receptor (BCR) signaling (Turner et al., 2000, supra). Syk kinase is also critical for tyrosine phosphorylation of multiple proteins which regulate important pathways leading from immunoreceptors, such as Ca.sup.2+ mobilization and mitogen-activated protein kinase (MAPK) cascades and degranulation. Syk kinase also plays a critical role in integrin signaling in neutrophils (see, e.g., Mocsai et al., 2002, Immunity 16:547-558).

As used herein, Syk kinase includes kinases from any species of animal, including but not limited to, homosapiens, simian, bovine, porcine, rodent, etc., recognized as belonging to the Syk family. Specifically included are isoforms, splice variants, allelic variants, mutants, both naturally occurring and man-made. The amino acid sequences of such Syk kinases are well known and available from GENBANK. Specific examples of mRNAs encoding different isoforms of human Syk kinase can be found at GENBANK accession no. gi|21361552|ref|NM.sub.-003177.2|, gi|496899|emb|Z29630.1|HSSYKPTK[496899] and gi|15030258|gb|BC011399.1|BC011399[15030258], which are incorporated herein by reference.

People having ordinary skill in the art will appreciate that tyrosine kinases belonging to other families may have active sites or binding pockets that are similar in three-dimensional structure to that of Syk. As a consequence of this structural similarity, such kinases, referred to herein as “Syk mimics,” are expected to catalyze phosphorylation of substrates phosphorylated by Syk. Thus, it will be appreciated that such Syk mimics, signal transduction cascades in which such Syk mimics play a role, and biological responses effected by such Syk mimics and Syk mimic-dependent signaling cascades may be regulated, and in particular inhibited, with many of the prodrugs described herein.

“Treating” or “treatment” as used herein concerns treatment of a disease or condition of interest in a patient or subject, particularly a human having the disease or condition of interest, and includes by way of example, and without limitation:

(i) preventing the disease or condition from occurring in a patient or subject, in particular, when such patient or subject is predisposed to the condition but has not yet been diagnosed as having it;

(ii) inhibiting the disease or condition, for example, arresting or slowing its development;

(iii) relieving the disease or condition, for example, causing diminution of a symptom or regression of the disease or condition or a symptom thereof; or

(iv) stabilizing the disease or condition.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

The above definitions and the following general formulas are not intended to include impermissible substitution patterns (e.g., methyl substituted with 5 fluoro groups). Such impermissible substitution patterns are easily recognized by a person having ordinary skill in the art.

### II. Introduction

The FDA approved four drugs in 2017 for treating AML: midostaurin (also known as RYDAPT®), gemtuzumab ozogamicin (also known as MYLOTARG®), enasidenib (also known as IDHIFA®), and a combined therapy of cytarabine and daunorubicin (also known as VYXEOS® or CPX-351). Midostaurin is an inhibitor of FLT-3 (FMS-like tyrosine kinase 3) as well as c-KIT, VEGFR-2 and PDGF. See Stein and Tallman, Curr. Cancer Drug Targets, 2012 June; 12(5): 522-530. VYXEOS® (or CPX-351) is a liposomal carrier containing cytarabine and daunorubicin in a fixed 5:1 ratio. Id. Gemtuzumab ozogamicin, a mixed lineage leukemia (MLL) translocation inhibitor, is an anti CD-33 monoclonal antibody linked to a cytotoxic agent. Id.

In 2018, the FDA approved ivosidenib (also known as TIBSOVO®), a small molecule inhibitor of IDH1 (isocitrate dehydrogenase isozyme 1), for relapsed or refractory acute myeloid leukemia. Approval was based on an open-label, single-arm, multicenter clinical trial (AG120-C-001, NCT02074839) that included 174 adult patients with relapsed or refractory AML with an IDH1 mutation. https://www.fda.gov/Drugs/InformationOnDrugs/ApprovedDrugs/ucm614128.htm.

Pratz et al. reported on a phase 1b/2 study of TAK-659, a dual FLT-3 and Syk inhibitor in patients with relapsed or refractory AML. Blood (2017) 130: 2622.

Walker et al. reported the results of a phase 1b/2 study of entospletinib (GS-9973) in treating AML as monotherapy and in combination with chemotherapy. Blood (2016) 128: 2831.

Hills et al. reported quizartinib (an FLT3 inhibitor) significantly improved overall survival in FLT3-ITD positive AML patients relapsed after stem cell transplantation or after failure of salvage chemotherapy. Blood (2015) 126: 2557.

Puissant et al. determined that FLT3 is transactivated by Syk via direct binding and that Syk is highly expressed in AML. Cancer Cell. 2014 Feb. 10; 25(2): 226-242.

Syk is a non-receptor protein-tyrosine kinase (PTK) that mediates inflammatory responses. Geahlen R L (2014), Trends Pharmacol Sci 35(8):414-422. PTKs, like Syk, are part of receptor-mediated signal transduction cascades that require their intracellular association with integral membrane receptors including toll-like receptors (TLRs (Han C, Jin J, Xu S et al (2010) Nat Immunol 11(8):734-742)) and Fc receptors (FcγR (Huang Z-Y, Barreda D R, Worth R G et al., (2006) J Leukoc Biol 80(6):1553-1562.), FcεRI (Lin K-C, Huang D-Y, Huang D-W et al (2016) J Mol Med (Berl) 94(2):183-194)). See also Valent et al., 2002, Intl. J Hematol. 75(4):257-362.

High pSyk (phosphorylated Syk) is associated with adverse outcomes in AML patients. Mohr et al., Cancer Cell. 2017 Apr. 10; 31(4): 549-562.

Multiple groups have observed anti-leukemic effects of Syk inhibitors, such as those disclosed herein, in cell lines, mouse-derived leukemias, and primary human leukemia, both ex vivo and in xenografts. Friedberg et al., “Inhibition of Syk with fostamatinib disodium has significant clinical activity in non Hodgkin's lymphoma and chronic lymphocytic leukemia” Blood 2010 Apr. 1; 1 15(13):2578-2585. Suljagic et al., “The Syk inhibitor fostamatinib disodium (R788) inhibits tumor growth in the Emu-TCLl transgenic mouse model of CLL by blocking antigen-dependent B-cell receptor signaling” Blood 2010 Dec. 2; 116(23):4894-905. Sharman J, Hawkins et al., “An open-label phase 2 trial of entospletinib (also known as GS-9973), a selective spleen tyrosine kinase inhibitor, in chronic lymphocytic leukemia” Blood 2015; 125(15):2336-43.

Early Phase I clinical trials are observing signals, perhaps biased to MLL, which is characterized by the presence of MLL fusion proteins that are the result of chromosomal translocations affecting the MLL gene at 11q23.

### III. Composition, Formulation, and Method Embodiments

As disclosed herein, the present inventors determined that segment-specific treatments would be beneficial in some types of AML. The inventors of the present disclosure have determined that 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or prodrugs thereof (e.g., prodrugs described below) would be an effective vehicle for treating AML, particularly in different types of patient populations, such as older patients who often experience toxic effects and/or difficulties with treatments using other types of treatments/therapeutics currently used for treating AML. Without being limited to a single theory, it currently is believed that 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can function as Syk inhibitors and can serve as effective therapeutics for AML, particularly because of Syk's transactivation of FLT3. The inventors also have determined that this compound (including any prodrugs) can be combined with additional therapies and/or therapeutic agents, such as those described herein.

Fostamatinib disodium hexahydrate, sold under the brand name TAVALISSE™ (and having a chemical name of disodium (6-[[5-fluoro-2-(3,4,5-trimethoxyanilino)pyrimidin-4-yl]amino]-2,2-dimethyl-3-oxo-pyrido[3,2-b][1,4]oxazin-4-yl)methyl phosphate hexahydrate) is a Syk kinase inhibitor and is a prodrug of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one. It was approved by the U.S. Food and Drug Administration in 2018 for the treatment of chronic immune thrombocytopenia (ITP). Fostamatinib disodium hexahydrate and other prodrug forms of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one are described in U.S. Pat. No. 7,449,458, the relevant portion of which is incorporated herein by reference. Other active prodrugs of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one can include, but are not limited to, 6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl dihydrogen phosphate (also known as fostamatinib) and sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate. Structures of certain of these compounds are illustrated below. While these are exemplary prodrugs, other prodrugs having different progroups and/or different counterions included in any such progroups (e.g., counterions other than sodium) also are contemplated.

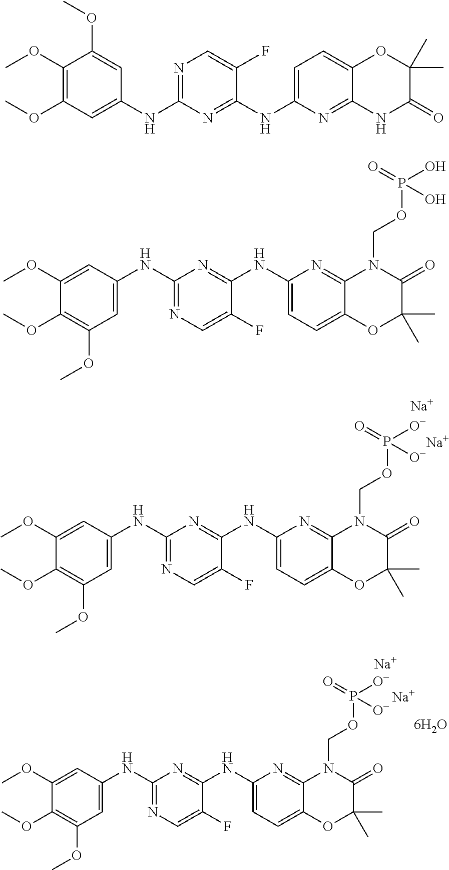

Disclosed herein are embodiments of a pharmaceutical composition comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof (e.g., fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or other prodrugs) and a second therapeutic agent. In some embodiments, the second therapeutic agent can be selected from any therapeutic agents disclosed herein, such as, but not limited to, daunorubicin, idarubicin, midostaurin, cytarabine, histamine dihydrochloride (typically in combination with interleukin 2), daunorubicin in combination with cytarabine, gemtuzumab ozogamicin, enasidenib, ivosidenib, TAK-659, entospletinib, quizartinib, gilteritinib, venetoclax, fludarabine, azacitidine, topotecan, arsenic trioxide, cerubidine, cyclophosphamide, daunorubicin hydrochloride, glasdegib maleate, dexamethasone, doxorubicin hydrochloride, enasidenib mesylate, gilteritinib fumarate, idarubicin hydrochloride, mitoxantrone hydrochloride, thioguanine, vincristine sulfate, or any combination thereof.

Treatment of most cases of AML is usually divided into two chemotherapy (chemo) phases: (i) remission induction (often just called induction); and (ii) consolidation (post-remission therapy). Optionally, the AML patient's blood may be treated by leukapheresis to remove white blood and leukemia cells before chemotherapeutic treatment. Induction destroys most of the normal bone marrow cells as well as the leukemia cells. But remission induction usually does not destroy all the leukemia cells—a small number often remain. Without consolidation treatment, the leukemia is likely to return within several months.

The most common remission induction regimens for AML include treatment with cytarabine, most often given continuously for seven days through an intravenous (IV) line. An anthracycline drug, such as daunorubicin or idarubicin, is also given in a single IV dose on each of three days during the first week of treatment. This is sometimes called the “7+3” regimen. For people whose AML has a mutation in the FLT3 gene (about one-third of patients), midostaurin may be added to the 7+3 regimen. As disclosed herein, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be used in remission induction, either alone or in combination with one or more (e.g., two, or two or more) therapeutic agents, such as the AML therapeutic agents described in this specification, particularly those described in this paragraph.

Consolidation following induction remission is intended to destroy any remaining leukemia cells and help prevent relapse. Among the consolidation regimens are the following:
(a) multiple cycles of high-dose cytarabine (ara-C) chemo (sometimes known as HiDAC);
(b) allogeneic (donor) stem cell transplant;
(c) autologous stem cell transplant;
(d) 1 or 2 cycles of standard dose cytarabine, possibly along with idarubicin, daunorubicin, or mitoxantrone; and
(e) non-myeloablative stem cell transplant (mini-transplant).
   In some embodiments, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, may be used alone or in combination with any consolidation regimen, including any of those disclosed herein.

For people who are not eligible for a stem cell transplant, immunotherapy with a combination of histamine dihydrochloride (also known as Ceplene) and interleukin 2 (also known as Proleukin) after the completion of consolidation has been shown to reduce the absolute relapse risk by 14%, translating to a 50% increase in the likelihood of maintained remission. Brune et al., Blood (July 2006) 108 (1): 88-96. In some embodiments, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, may be used in this combined treatment as well.

In the method and composition embodiments of the present disclosure, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be combined or used in combination with an FLT3 inhibitor. In another embodiment, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be combined or used in combination with an IDH1 inhibitor. In yet additional embodiments, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be combined or used in combination with any recognized method of treating AML, such as with any FDA approved agent for treating AML, including midostaurin, cytarabine in combination with daunorubicin, gemtuzumab ozogamicin, enasidenib, ivosidenib, or any combination thereof. In some embodiments, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, may also be used in combination with TAK-659, entospletinib, quizartinib, or any combination thereof. In exemplary embodiments, fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or the like is used in combination with daunorubicin, idarubicin, midostaurin, cytarabine, histamine dihydrochloride (typically in combination with interleukin 2), daunorubicin in combination with cytarabine, gemtuzumab ozogamicin, enasidenib, ivosidenib, fludarabine, azacitidine, topotecan, or any combination thereof. In yet additional exemplary embodiments, fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or the like is used in combination with TAK-659, entospletinib, quizartinib, or any combination thereof.

6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, also can be combined or used with other chemotherapeutic agents, such nucleoside analogues (e.g., fludarabine, which also is known as FLUDARA®; and azacitidine, which also is known as VIDAZA®) and topoisomerase inhibitors (e.g., topotecan, which also is known as HYCAMTIN®).

In some embodiments, any chemotherapeutic treatments disclosed herein may optionally be combined with radiation.

Pharmaceutical composition embodiments comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be manufactured by means of conventional mixing, dissolving, granulating, dragee-making, levigating, emulsifying, encapsulating, entrapping, and/or lyophilization processes. Pharmaceutical composition embodiments can be formulated in conventional manner using one or more physiologically acceptable carriers, diluents, excipients, or auxiliaries that facilitate processing the active compounds into preparations that can be used pharmaceutically.

6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, and pharmaceutical formulations and/or pharmaceutical compositions thereof, can be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients and vehicles appropriate for each route of administration.

Pharmaceutical composition embodiments comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, may conveniently be presented in dosage unit form and can be prepared by any of the methods well known in the art of pharmacy. The pharmaceutical compositions can be, for example, prepared by uniformly and intimately bringing the active ingredient into association with a liquid carrier or a finely divided solid carrier or both, and then, if necessary, shaping the product into the desired formulation. In some embodiments, the active object compound is included in an amount sufficient to produce the desired therapeutic effect. For example, pharmaceutical compositions of the present disclosure may take a form suitable for virtually any mode of administration, including, for example, topical, ocular, oral, buccal, systemic, nasal, injection, transdermal, rectal, vaginal, etc., or a form suitable for administration by inhalation or insufflation.

For topical use, creams, ointments, jellies, gels, solutions, suspensions (or the like) containing 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be employed. In certain embodiments, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be formulated for topical administration with polyethylene glycol (PEG). In some embodiments, topical use formulations may optionally comprise additional pharmaceutically acceptable ingredients such as diluents, stabilizers, and/or adjuvants.

According to the present disclosure, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be used for manufacturing a composition or medicament, including medicaments suitable for topical administration. The present disclosure also discloses methods for manufacturing compositions including 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, in a form that is suitable for topical administration.

In yet some additional embodiments, systemic formulations comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, are described. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable formulation embodiments include sterile suspensions, solutions or emulsions of the active compound(s) in aqueous or oily vehicles. The injectable formulation may also contain formulating agents, such as suspending, stabilizing, and/or dispersing agents. The injectable formulation can be presented in unit dosage form, e.g., in ampules or in multidose containers, and may contain added preservatives.

In some additional embodiments, the injectable formulation can be provided in powder form for reconstitution with a suitable vehicle, including but not limited to sterile, pyrogen-free water, buffer, dextrose solution, etc., before use. To this end, the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be dried by any suitable technique known in the art with the benefit of the present disclosure, such as lyophilization, and then reconstituted prior to use.

Also disclosed are transmucosal formulation embodiments comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof. For transmucosal administration, penetrants appropriate to the barrier to be permeated are used in the formulation. Such penetrants are known in the art and can be selected with the benefit of the present disclosure.

Also disclosed herein are embodiments of an oral formulation comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof. For oral administration, the pharmaceutical composition embodiments disclosed herein may take the form of, for example, lozenges, tablets, or capsules prepared by conventional means with pharmaceutically acceptable excipients, such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); wetting agents (e.g., sodium lauryl sulfate); or any combination thereof. The tablets can be coated by methods well known in the art with the benefit of the present disclosure and can be coated with, for example, sugars, films, or enteric coatings. Inactive ingredients include mannitol, sodium bicarbonate, sodium starch glycolate, povidone, and magnesium stearate, any or all of which, when the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one (or a prodrug thereof) formulation is in tablet form, can be in the tablet core. Tablets may also be film coated, and the file coating can comprise one or more of polyvinyl alcohol, titanium dioxide, polyethylene glycol 3350, talc, iron oxide yellow, and iron oxide red.

Additionally, the pharmaceutical composition embodiments containing the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, as an active ingredient in a form suitable for oral use, may also include, for example, troches, lozenges, aqueous or oily suspensions, dispersible powders or granules, emulsions, hard or soft capsules, or syrups, or elixirs. Compositions intended for oral use can be prepared according to any method known to the art with the benefit of the present disclosure for manufacturing pharmaceutical compositions and such compositions may contain one or more agents selected from sweetening agents, flavoring agents, coloring agents, preserving agents, or any combination thereof, to provide pharmaceutically elegant and palatable preparations. In some embodiments, tablets can contain the active ingredient in admixture with non-toxic pharmaceutically acceptable excipients that are suitable for manufacturing tablets. These excipients can be for example, inert diluents, such as calcium carbonate, sodium carbonate, lactose, calcium phosphate, or sodium phosphate; granulating and disintegrating agents (e.g., corn starch, or alginic acid); binding agents (e.g. starch, gelatin or acacia); lubricating agents (e.g. magnesium stearate, stearic acid or talc); or any combination thereof. The tablets can be uncoated or they can be coated by known techniques to delay disintegration and absorption in the gastrointestinal tract and thereby provide a sustained action over a longer period. For example, a time delay material, such as glyceryl monostearate or glyceryl distearate, can be employed. They may also be coated by the techniques described in the U.S. Pat. Nos. 4,256,108; 4,166,452; and U.S. Pat. No. 4,265,874 to form osmotic therapeutic tablets for control release. The pharmaceutical compositions of the present disclosure may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means by combining 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); preservatives (e.g., methyl or propyl-p-hydroxybenzoates or sorbic acid); or any combinations thereof. The preparations may also contain buffer salts, preservatives, flavoring, coloring and/or sweetening agents as appropriate.

Preparations for oral administration can be suitably formulated to give controlled release of the active compound or prodrug, as is well known.

For buccal administration, the compositions may take the form of tablets or lozenges formulated in conventional manner.

For rectal and vaginal routes of administration, the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be formulated as solutions (for retention enemas), suppositories, or ointments containing conventional suppository bases, such as cocoa butter or other glycerides.

For nasal administration or administration by inhalation or insufflation, the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be conveniently delivered in the form of an aerosol spray from pressurized packs or a nebulizer with the use of a suitable propellant, such as dichlorodifluoromethane, trichlorofluoromethane, dichlorotetrafluoroethane, fluorocarbons, carbon dioxide, or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, and a suitable powder base, such as lactose or starch.

The pharmaceutical compositions comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be in the form of a sterile injectable aqueous or leaginous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution and isotonic sodium chloride solution.

According to the present disclosure, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be used for manufacturing a composition or medicament, including medicaments suitable for rectal or urethral administration. The present disclosure also relates to method embodiments for manufacturing compositions comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, in a form that is suitable for urethral or rectal administration, including suppositories.

According to the present disclosure, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can also be delivered by any of a variety of inhalation devices and methods known in the art, including, for example: U.S. Pat. Nos. 6,241,969; 6,060,069; 6,238,647; 6,335,316; 5,364,838; 5,672,581; WO96/32149; WO95/24183; U.S. Pat. Nos. 5,654,007; 5,404,871; 5,672,581; 5,743,250; 5,419,315; 5,558,085; WO98/33480; U.S. Pat. Nos. 5,364,833; 5,320,094; 5,780,014; 5,658,878; 5,518,998; 5,506,203; 5,661,130; 5,655,523; 5,645,051; 5,622,166; 5,577,497; 5,492,112; 5,327,883; 5,277,195; U.S. Publication No. 20010041190; U.S. Publication No. 20020006901; and U.S. Publication No. 20020034477.

Included among the devices that can be used to administer 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, are those well-known in the art, such as, metered dose inhalers, liquid nebulizers, dry powder inhalers, sprayers, thermal vaporizers, and the like. Other suitable administration technologies include electrohydrodynamic aerosolizers.

In addition, the inhalation device is preferably practical, in the sense of being easy to use, small enough to carry conveniently, capable of providing multiple doses, and durable. Some specific examples of commercially available inhalation devices are Turbohaler (Astra, Wilmington, Del.), Rotahaler (Glaxo, Research Triangle Park, N.C.), Diskus (Glaxo, Research Triangle Park, N.C.), the Ultravent nebulizer (Mallinckrodt), the Acorn II nebulizer (Marquest Medical Products, Totowa, N.J.) the Ventolin metered dose inhaler (Glaxo, Research Triangle Park, N.C.), or the like. In one embodiment, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be delivered by a dry powder inhaler or a sprayer.

The formulation of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, in the aerosol. For example, shorter periods of administration can be used at higher concentrations 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, in the nebulizer solution. Devices such as metered dose inhalers can produce higher aerosol concentrations, and can be operated for shorter periods to deliver the desired amount of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, in some embodiments. Devices such as dry powder inhalers deliver active agent until a given charge of agent is expelled from the device. In this type of inhaler, the amount of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, in a given quantity of the powder determines the dose delivered in a single administration. The formulation of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, is selected to yield the desired particle size in the chosen inhalation device.

Formulations of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, for administration from a dry powder inhaler may typically include a finely divided dry powder containing the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the prodrug thereof, but the powder can also include a bulking agent, buffer, carrier, excipient, another additive, or the like. Additives can be included in a dry powder formulation of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like.

In some embodiments, pharmaceutical composition embodiments comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, suitable for administration by inhalation are disclosed. 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be used for manufacturing a composition or medicament, including medicaments suitable for administration by inhalation. Also disclosed are embodiments of a method for manufacturing compositions including 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, in a form that is suitable for administration, including administration by inhalation. For example, a dry powder formulation can be manufactured in several ways, using conventional techniques, such as described in any of the publications mentioned above and incorporated expressly herein by reference, and for example, U.S. Pat. No. 5,700,904, the entire disclosure of which is incorporated expressly herein by reference. Particles in the size range appropriate for maximal deposition in the lower respiratory tract can be made by micronizing, milling, or the like. And a liquid formulation can be manufactured by dissolving the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, in a suitable solvent, such as water, at an appropriate pH, including buffers or other excipients.

For ocular administration, the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be formulated as a solution, emulsion, suspension, etc. suitable for administration to the eye. A variety of vehicles suitable for administering compounds to the eye are known in the art and can be selected with the benefit of the present disclosure. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851.

For prolonged delivery, the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch that slowly releases the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, for percutaneous absorption can be used. To this end, permeation enhancers can be used to facilitate transdermal penetration of the active compound(s). Suitable transdermal patches are described in for example, U.S. Pat. No. 5,407,713.; U.S. Pat. Nos. 5,352,456; 5,332,213; 5,336,168; 5,290,561; 5,254,346; 5,164,189; 5,163,899; 5,088,977; 5,087,240; 5,008,110; and 4,921,475.

Alternatively, other pharmaceutical delivery systems can be employed for pharmaceutical composition embodiments disclosed herein, such as liposomes and emulsions. Certain organic solvents such as dimethylsulfoxide (DMSO) may also be employed in some embodiments.

When used in combination with additional therapeutic agents (e.g., the chemotherapeutic agents described herein for treating AML), 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, may be administered sequentially or simultaneously, in the same or separate dosage units, in the same or different forms. For example, in one embodiment, the pharmaceutical composition of the present disclosure comprises 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, and one or more (e.g., two, or two or more) additional active agents for treating AML (e.g., the active agents for treating AML described herein). The amount of the other AML active agents can be easily and routinely determined using art recognized methods. In one embodiment, the other active agents are present in the composition in the amount, form, and regimen typically used for the respective agent, such as those approved by the FDA for the respective agent. Such amounts, forms, and regimens can be adjusted using art recognized methods to account for co-treatment with 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof.

The pharmaceutical compositions may, if desired, be presented in a pack or dispenser that may contain one or more unit dosage forms containing the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, and/or any second therapeutic agent. The pack may, for example, comprise metal or plastic foil, such as a blister pack. The pack or dispenser device can be accompanied by instructions for administration.

6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, will generally be used in an amount effective to provide a therapeutic benefit in the treatment of AML. By therapeutic benefit is meant eradication or amelioration of AML and/or eradication or amelioration of one or more of the symptoms associated with AML such that the patient reports an improvement in feeling or condition, notwithstanding that the patient may still be afflicted with AML. Therapeutic benefit also includes halting or slowing the progression of the disease, regardless of whether improvement is realized.

The amount of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, administered will depend upon a variety of factors, including, for example, the mode of administration, the severity of the condition being treated and the age and weight of the patient, etc. Determination of an effective dosage is well within the capabilities of those having ordinary skill in the art with the benefit of the present disclosure.

As known by those of ordinary skill in the art, the preferred dosage of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, will also depend on the age, weight, general health and severity of the condition of the individual being treated. Dosage may also need to be tailored to the sex of the individual and/or where administered by inhalation, the lung capacity of the individual. Dosage may also be tailored to individuals suffering from more than one condition or those individuals who have additional conditions which affect lung capacity and the ability to breathe normally, for example, emphysema, bronchitis, pneumonia, respiratory infections, etc. Dosage, and frequency of administration of the compounds or prodrugs thereof, will also depend on whether the compounds are formulated for treatment of acute episodes of thrombocytopenia or for the prophylactic treatment of such a disorder. A person of ordinary skill in the art will be able to determine the optimal dose for a particular individual, particularly with the benefit of the present disclosure.

Effective dosages can be estimated initially from in vitro assays. For example, an initial dosage for use in animals can be formulated to achieve a circulating blood or serum concentration of active compound that is at or above an IC₅₀ of the particular compound as measured in as in vitro assay. Calculating dosages to achieve such circulating blood or serum concentrations taking into account the bioavailability of the particular compound is well within the capabilities of those having ordinary skill in the art, particularly with the benefit of the present disclosure. For guidance, the reader is referred to Fingl & Woodbury, “General Principles,” In: Goodman and Gilman's The Pharmaceutical Basis of Therapeutics, Chapter 1, 13th edition, Pergamon Press, 2017, and the references cited therein.

Initial dosages can also be estimated from in vivo data, such as animal models. Animal models useful for testing the efficacy of compounds to treat or prevent the various diseases described above are well-known in the art.

Dosage amount and interval can be adjusted individually to provide plasma levels of the compound that is sufficient to maintain therapeutic effect. For example, 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, can be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, may not be related to plasma concentration. A person of ordinary skill in the art will be able to optimize effective local dosages without undue experimentation and particularly with the benefit of the present disclosure.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration and various factors discussed above. It is contemplated that a typical dosage when used alone or when co-administered with another chemotherapeutic agent will range from about 0.001 mg/kg to about 1000 mg/kg, about 0.01 mg/kg to about 100 mg/kg, or from about 0.1 mg/kg to about 10 mg/kg.

Typical daily administrations are in the range of 100-400 mg/day, e.g., 100, 150, 200, 250, 300, 350, or 400 mg/day. Administration can be once or twice daily, e.g., 100, 150, or 200 mg BID. Accordingly, pharmaceutical dosage forms comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, may contain from 50-400 mg 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the prodrug thereof, e.g., 50, 100, 150, 200, 250, 300, 350, or 400 mg 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the prodrug thereof.

In one embodiment, the amount of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the prodrug thereof, in a composition to be administered, or the amount of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the prodrug thereof, to be administered in a method disclosed herein, is a suboptimal dose.

The 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, pharmaceutical compositions described herein for use in treating AML can comprise 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, as the sole active agent or may further comprise one or more (e.g., two, or two or more) additional therapeutic agents. In some embodiments, the additional therapeutic agent(s) are chemotherapeutic agents useful for treating AML, e.g., the therapeutic agents described herein.

As described herein, prodrugs of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one can be used. Such prodrugs are usually, but need not be, pharmacologically inactive until converted into their active drug form. Indeed, at least some of the compounds described herein include promoieties that are hydrolyzable or otherwise cleavable under conditions of use. For example, ester groups commonly undergo acid-catalyzed hydrolysis to yield the parent carboxylic acid when exposed to the acidic conditions of the stomach or base-catalyzed hydrolysis when exposed to the basic conditions of the intestine or blood. Thus, when administered to a subject orally, compounds that include ester moieties can be considered prodrugs of their corresponding carboxylic acid, regardless of whether the ester form is pharmacologically active.

The mechanism by which the progroups metabolize is not critical and can be caused, for example, by hydrolysis under the acidic conditions of the stomach, as described above, and/or by enzymes present in the digestive tract and/or tissues or organs of the body. Indeed, the progroup(s) can be selected to metabolize at a particular site within the body. For example, many esters are cleaved under the acidic conditions found in the stomach. Prodrugs designed to cleave chemically in the stomach to the active compounds can employ progroups including such esters. Alternatively, the progroups can be designed to metabolize in the presence of enzymes such as esterases, amidases, lipolases, and phosphatases, including ATPases and kinase, etc. Progroups including linkages capable of metabolizing in vivo are well known and include, by way of example and not limitation, ethers, thioethers, silylethers, silylthioethers, esters, thioesters, carbonates, thiocarbonates, carbamates, thiocarbamates, ureas, thioureas, and carboxamides. In some instances, a “precursor” group that is oxidized by oxidative enzymes such as, for example, cytochrome P₄₅₀ of the liver, to a metabolizable group, can be selected.

In the prodrugs, any available functional moiety can be masked with a progroup to yield a prodrug. Functional groups within the disclosed compounds that can be masked with progroups for inclusion in a promoiety include, but are not limited to, amines (primary and secondary), hydroxyls, sulfanyls (thiols), and carboxyls. A wide variety of progroups, as well as the resultant promoieties, suitable for masking functional groups in active compounds to yield prodrugs are well-known in the art. For example, a hydroxyl functional group can be masked as a sulfonate, ester, or carbonate promoiety, which can be hydrolyzed in vivo to provide the hydroxyl group. An amino functional group can be masked as an amide, carbamate, imine, urea, phosphenyl, phosphoryl, or sulfenyl promoiety, which can be hydrolyzed in vivo to provide the amino group. A carboxyl group can be masked as an ester (including silyl esters and thioesters), amide, or hydrazide promoiety, which can be hydrolyzed in vivo to provide the carboxyl group. In some embodiments, the progroup is a phosphate-containing progroup of the formula —(CR^{d}R^{d})O—P(O)(OH)(OH), or a salt thereof, y is an integer ranging from 1 to 3, typically 1 or 2; and each R^{d} is, independently of the others, selected from hydrogen, substituted or unsubstituted lower alkyl, substituted or unsubstituted phenyl, substituted or unsubstituted methyl and substituted or unsubstituted benzyl. In a specific embodiment, each R^{d} is, independently of the others, selected from hydrogen and unsubstituted lower alkyl. Specific exemplary phosphate-containing progroups include —CH₂—O—P(O)(OH)(OH) and —CH₂CH₂—O—P(O)(OH)(OH) and/or the corresponding salts (e.g., —CH₂—O—P(O)(ONa)₂ and —CH₂CH₂—O—P(O)(ONa)₂). Other specific examples of suitable progroups and their respective promoieties will be apparent to those having ordinary skill in the art with the benefit of the present disclosure. All of these progroups, alone or in combinations, can be included in the prodrugs. In particular embodiments, prodrugs of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one are selected from, but are not limited to, fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or the like.

Also disclosed herein are embodiments of a method of using compound embodiments and pharmaceutical composition embodiments of the present disclosure to treat AML. In some embodiments, the method comprises administering a therapeutically effective amount of (i) 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a phosphate-containing prodrug thereof, and (ii) a second therapeutic agent to a subject, wherein the subject has, or is at risk of developing, acute myeloid leukemia (AML). In some embodiments, the second therapeutic agent is administered simultaneously with the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof. In yet some additional embodiments, the second therapeutic agent is administered sequentially with the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof. In some embodiments, the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof; the second therapeutic agent; or both of these components are administered in a suboptimal dose.

In yet some additional embodiments, the method comprises identifying a subject that has, or is at risk of developing, acute myeloid leukemia (AML); and treating the subject with a pharmaceutical composition comprising (i) 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof, and (ii) a second therapeutic agent. In particular embodiments, identifying a subject that has, or is at risk of developing, acute myeloid leukemia (AML) comprises identifying a subject having an FLT3-ITD mutation, a high Syk activity, or a combination thereof. In some such embodiments, the second therapeutic agent can be an FLT3 inhibitor.

### IV. Kit Embodiments

Also provided are kits for administration of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, alone or together with one or more (e.g., two, or two or more) additional therapeutic agents. The kit may include a dosage amount of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof. Kits may further comprise suitable packaging and/or instructions for use of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, and, optionally, one or more (e.g., two, or two or more) additional chemotherapeutic agents for treating AML. The instructions can be in any suitable tangible format, including, but not limited to, printed matter, videotape, computer readable disk, or optical disc.

Kits may also comprise a means for the delivery of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, or compositions thereof, such as an inhaler, spray dispenser (e.g. nasal spray), syringe for injection or pressure pack for capsules, tables, suppositories, or other device as described herein. The kits may also comprise similar contents for one or more (e.g., two, or two or more) other therapeutic agents for treating AML.

The kits may contain a single dosage, a daily dosage, or sufficient dosages of the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a prodrug thereof, or composition thereof to provide effective treatment for an individual for an extended period, such as a week, 2 weeks, 3 weeks, 4 weeks, 6 weeks or 8 weeks or more. The dosage may be a suboptimal dose.

In another embodiment, the kits are for treating an individual who suffers from AML.

### V. Overview of Several Embodiments

Disclosed herein are embodiments of a method, comprising administering a therapeutically effective amount of (i) 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a phosphate-containing prodrug thereof, and (ii) a second therapeutic agent to a subject, wherein the subject has, or is at risk of developing, acute myeloid leukemia (AML).

In any or all embodiments, the phosphate-containing prodrug is fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or a combination thereof.

In any or all of the above embodiments, the second therapeutic agent is a chemotherapeutic agent that inhibits DNA synthesis, topoisomerase, FLT3, IDH1, or Syk.

In any or all of the above embodiments, the second therapeutic agent is daunorubicin; idarubicin; midostaurin; cytarabine; histamine dihydrochloride, alone or in combination with interleukin 2; daunorubicin in combination with cytarabine; gemtuzumab ozogamicin; enasidenib; ivosidenib; TAK-659; entospletinib; quizartinib; gilteritinib; venetoclax; fludarabine; azacitidine; topotecan; or any combination thereof.

In any or all of the above embodiments, the second therapeutic agent is quizartinib.

In any or all of the above embodiments, the second therapeutic agent is administered simultaneously with the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof.

In any or all of the above embodiments, the second therapeutic agent is administered sequentially with the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof.

In any or all of the above embodiments, the (i) the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof, (ii) the second therapeutic agent, or both (i) and (ii) are administered in a suboptimal dose.

Also disclosed herein are embodiments of a pharmaceutical composition, comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a phosphate-containing prodrug thereof, in an amount effective to treat acute myeloid leukemia (AML).

In any or all embodiments, the phosphate-containing prodrug is fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or a combination thereof.

In any or all of the above embodiments, the amount of the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof is a suboptimal dose.

In any or all of the above embodiments, the pharmaceutical composition further comprises a second therapeutic agent present in an amount effective to treat AML.

In any or all of the above embodiments, the amount of the second therapeutic agent is a suboptimal dose.

In any or all of the above embodiments, the amount of the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof is a suboptimal dose.

In any or all of the above embodiments, the second therapeutic agent is a chemotherapeutic agent that inhibits DNA synthesis, topoisomerase, FLT3, IDH1, or Syk.

In any or all of the above embodiments, the second therapeutic agent is daunorubicin; idarubicin; midostaurin; cytarabine; histamine dihydrochloride, alone or in combination with interleukin 2; daunorubicin in combination with cytarabine; gemtuzumab ozogamicin; enasidenib; ivosidenib; TAK-659; entospletinib; quizartinib; gilteritinib; venetoclax; fludarabine; azacitidine; topotecan; or any combination thereof.

In any or all of the above embodiments, the second therapeutic agent is quizartinib.

Also disclosed herein are embodiments of a kit, comprising: 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a phosphate-containing prodrug thereof; and instructions for treating AML with the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof.

In any or all embodiments, the phosphate-containing prodrug is fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or a combination thereof.

In any or all of the above embodiments, the amount of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof, in the kit is a suboptimal dose.

In any or all of the above embodiments, the kit further comprises a second therapeutic agent for treating AML.

In any or all of the above embodiments, the second therapeutic agent is present at a suboptimal dose.

In any or all of the above embodiments, the amount of the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof in the kit is a suboptimal dose.

In any or all of the above embodiments, the second therapeutic agent is a chemotherapeutic agent that inhibits DNA synthesis, topoisomerase, FLT3, IDH1, or Syk.

In any or all of the above embodiments, the second therapeutic agent is daunorubicin; idarubicin; midostaurin; cytarabine; histamine dihydrochloride, alone or in combination with interleukin 2; daunorubicin in combination with cytarabine; gemtuzumab ozogamicin; enasidenib; ivosidenib; TAK-659; entospletinib; quizartinib; gilteritinib; venetoclax; fludarabine; azacitidine; topotecan; or any combination thereof.

In any or all of the above embodiments, the second therapeutic agent is quizartinib.

Also disclosed herein are embodiments of a method, comprising: identifying a subject that has, or is at risk of developing, acute myeloid leukemia (AML); and treating the subject with a pharmaceutical composition comprising (i) 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof, and (ii) a second therapeutic agent.

In any or all embodiments, the second therapeutic agent is selected from daunorubicin; idarubicin; midostaurin; cytarabine; histamine dihydrochloride, alone or in combination with interleukin 2; daunorubicin in combination with cytarabine; gemtuzumab ozogamicin; enasidenib; ivosidenib; TAK-659; entospletinib; quizartinib; gilteritinib; venetoclax; fludarabine; azacitidine; topotecan; or any combination thereof.

In any or all of the above embodiments, identifying a subject that has, or is at risk of developing, acute myeloid leukemia (AML) comprises identifying a subject having an FLT3-ITD mutation.

In any or all of the above embodiments, the second therapeutic agent is an FLT3 inhibitor.

In any or all of the above embodiments, identifying a subject that has, or is at risk of developing, acute myeloid leukemia (AML) comprises identifying a subject having high Syk activity.

### VI. Examples

### Example 1

About 50 patients with relapsed/refractory AML (but not with RAS mutations) undergo deep genomic analysis. Ex vivo drug sensitivity studies in plasma and bank bone marrow samples are conducted.

All patients receive fostamatinib as single agent, 250 mg bid, decreasing to 200 mg bid after 4 weeks. Bone marrow biopsies are taken at 2 weeks, and if a decrease in bone marrow blast percentage is observed, fostamatinib is administered as a single agent for two additional weeks. If no response is observed at 2 weeks, or no complete response at 4 weeks after anoty bone marrow biopsy, then fostamatinib is co-administered with quizartinib (150 mg bid, with increase to 200 bid after 4 weeks if well-tolerated).

The overall response (CR+CRi+PR) of combination at 4, 8, 12 weeks then every 8 weeks is assessed.

### Example 2

In this example, the ability of spleen-derived murine primary cells expressing FLT3-ITD+Syk or FLT3-ITD+Syk-TEL are injected (tail vein) as a secondary transplant into sublethally irradiated (400 cGy) male BALB/c mice (age=6-weeks-old). The following daily treatments are administered by oral gavage when the white blood cell count reaches a suitable level (e.g., ˜30 K/μl): (i) 80 mg/kg fostamatinib; (ii) a vehicle control (e.g., 35% TPGS, 60% PEG 400, 5% propylene glycol; in 100 μl; n=6 per condition); or (iii) a combination of fostamatinib and another therapeutic agent (e.g., daunorubicin, idarubicin, midostaurin, cytarabine, histamine dihydrochloride (typically in combination with interleukin 2), daunorubicin in combination with cytarabine, gemtuzumab ozogamicin, enasidenib, ivosidenib, TAK-659, entospletinib, quizartinib, gilteritinib, venetoclax, fludarabine, azacitidine, topotecan, or any combination thereof) are administered by oral gavage. Syk and/or FLT3 inhibition can then be measured using a method known to a person of ordinary skill in the art with the benefit of the present disclosure.

### Example 3

Isolation of bone marrow mononuclear cells from human bone marrow aspirates can be achieved by Ficoll density centrifugation (400×g, RT, 45 min). CD34+ bone marrow mononuclear cells can be isolated using the human CD34 MultiSort Kit (Miltenyi Biotec). In some embodiments, bone marrow mononuclear cells are washed (2×) with MACS buffer (PBS containing 0.5% bovine serum albumin (BSA) and 2 mM EDTA). The cells can be passed through a mesh to remove cell clumps and can then be incubated in MACS buffer, which can be supplemented with FcR Blocking Reagent, and CD34 MultiSort MicroBeads for a particular time and at a particular temperature (e.g., 30 minutes at 4° C.). Excess beads are removed by washing and then magnetic separation using LS columns is carried out. After elution, cells are washed once in MACS buffer and can be resuspended in SFEM supplemented with diverse cytokines. Flow cytometry can be used to assess the purity of the isolated cells. For apoptosis measurements, cells (e.g., 2×10⁴) are seeded in culture medium (e.g., 100 μl) and treated with DMSO (as a control), 1 μM, 5 μM and 10 μM 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one for 24 hours. The Annexin V Apoptosis Detection Kit I (BD Bioscience) can be used to detect apoptotic cell death. Cells are harvested and washed once with PBS, and can be resuspended in Annexin V binding buffer (200 μl 1×) containing 5 μl Annexin-PE or Annexin-APC and incubated at room temperature in the dark. After washing (e.g., once with 1× Annexin binding buffer), cells are resuspended in 1× Annexin V binding buffer containing 7-AAD (5 μl) and incubated at room temperature in the dark. 1× Annexin V binding buffer (200 μl) is added and cells are analysed using a BD LSRFortessa flow cytometer.

AML samples expressing high levels of HOXA9 and MEIS1 can exhibit increased expression of Syk and pSyk, and can be more sensitive to 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one (alone or in combination with one or more therapeutic agents described herein) as compared with samples with weak MEIS1 expression. Syk inhibition does not affect the viability of CD34⁺ progenitor cells isolated from healthy donors. And, Syk inhibition can significantly prolong survival of NSG mice that are transplanted with patient-derived AML cells that overexpress HOXA9 and MEIS1.

The effect of Syk inhibition in HOXA9 and HOXA9/MEIS1 cells by monitoring the fate of individual cells and their progeny by time-lapse microscopy and single-cell tracking also can be carried out. A significant increase can be observed in cell death in HOXA9/MEIS1 cells treated with 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one (compared with cells treated with DMSO) and/or a prodrug thereof (each alone or in combination with one or more therapeutic agents described herein).

### Example 4

In this example, mice transplanted with HOXA9 MEIS1 or HOXA9 cells are treated with fostamatinib using feed impregnated with fostamatinib at 3 g/kg, 5 g/kg or 8 g/kg (AIN-76A rodent diet). In some examples, one week of treatment with fostamatinib can reduce the percentage of leukemic cells in mice transplanted with HOXA9/MEIS1 cells. Treatment with fostamatinib for longer periods (e.g., 20 days) can significantly prolong the survival of mice transplanted with HOXA9/MEIS1 cells.

### Example 5

In this example, the ability of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one (or a prodrug thereof) to synergistically enhance cytotoxic activity of venetoclax in AML cell lines is evaluated. Cytotoxic effects of venetoclax can be determined using Annexin V/PI staining against a panel of different AML cell lines. At various concentrations (e.g., ranging up to 0.25 M), venetoclax, alone, exhibits only modest activity against most AML cell lines; however, treating the cells with 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one (or a prodrug thereof) at different concentrations for a suitable period of time (e.g., 2 hours) can markedly increase the cytotoxic activity of venetoclax. In some examples, a strong synergistic effect can be observed and in some examples, it can occur in the absence of any substantial cytotoxic effects of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one (or the prodrug thereof) on its own.

The activity of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one (or a prodrug thereof), alone, also can be evaluated. To do so, activity of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one can be tested in the appropriate mouse model xenografted with an AML cell line. Tumors can be injected subcutaneously (e.g., in the right flank) and allowed to grow until they reached an appropriate size (e.g., ≥250 mm³). Intraperitoneal injections can be used to introduce the treatment. 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one alone can partially inhibit tumor growth, but combining it with venetoclax can result in significantly greater activity and even potentially induce tumor regression. In some examples, significant differences in tumor volume between mice receiving 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one alone and mice receiving 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one in combination with venetoclax can be observed.

### Example 6

In this example, ex vivo functional screening is performed with isolated mononuclear cells from AML samples to assess composition activity. Cells are arrayed into well plates containing 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one and prodrugs thereof, such as fostamatinib. The panel can contain graded concentrations of the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one and any prodrugs thereof. Control wells (e.g., DMSO) and any positive-control wells are placed on each plate. Daughter/Designation plates can be created and used and can be sealed with thermal seals. Primary mononuclear cells are plated across the compound/inhibitor panels. Cells can be seeded into the well-plate assay plates at 10,000 cells per well in Roswell Park Memorial Institute (RPMI) 1640 medium, which can be supplemented with fetal bovine serum (FBS) (10%), 1-glutamine, penicillin-streptomycin, and β-mercaptoethanol (10-4 M). After three days of culture at 37° C. in 5% CO₂, MTS reagent (CellTiter96 AQueous One; Promega) can be added and the optical density can be measured at an appropriate wavelength, such as 490 nm. Raw absorbance values are adjusted to a reference blank value and then used to determine cell viability (normalized to untreated control wells).

In view of the many possible embodiments to which the principles of the present disclosure may be applied, it should be recognized that the illustrated embodiments are only preferred examples and should not be taken as limiting. Rather, the scope of the present disclosure is defined by the following claims. We therefore claim as our invention all that comes within the scope and spirit of these claims.

1. A method, comprising: administering a therapeutically effective amount of (i) 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a phosphate-containing prodrug thereof, and (ii) a second therapeutic agent to a subject, wherein the subject has, or is at risk of developing, acute myeloid leukemia (AML).

2. The method of claim 1, wherein the phosphate-containing prodrug is fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or a combination thereof.

3. The method according to claim 1, wherein the second therapeutic agent is a chemotherapeutic agent that inhibits DNA synthesis, topoisomerase, FLT3, IDH1, or Syk.

4. The method according to claim 1, wherein the second therapeutic agent is daunorubicin; idarubicin; midostaurin; cytarabine; histamine dihydrochloride, alone or in combination with interleukin 2; daunorubicin in combination with cytarabine; gemtuzumab ozogamicin; enasidenib; ivosidenib; TAK-659; entospletinib; quizartinib; gilteritinib; venetoclax; fludarabine; azacitidine; topotecan; arsenic trioxide; cerubidine; cyclophosphamide; daunorubicin hydrochloride; glasdegib maleate; dexamethasone; doxorubicin hydrochloride; enasidenib mesylate; gilteritinib fumarate; idarubicin hydrochloride; mitoxantrone hydrochloride; thioguanine; vincristine sulfate; or any combination thereof.

5. The method according to claim 4, wherein the second therapeutic agent is quizartinib.

6. The method according to any claim 1, wherein the second therapeutic agent is administered simultaneously with the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof.

7. The method according to claim 1, wherein the second therapeutic agent is administered sequentially with the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof.

8. The method according to claim 1, wherein (i) the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof, (ii) the second therapeutic agent, or both (i) and (ii) are administered in a suboptimal dose.

9. A pharmaceutical composition, comprising 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a phosphate-containing prodrug thereof, in an amount effective to treat acute myeloid leukemia (AML).

10. The pharmaceutical composition according to claim 9, wherein the phosphate-containing prodrug is fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or a combination thereof.

11. The pharmaceutical composition according to claim 9, wherein the amount of the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof is a suboptimal dose.

12. The pharmaceutical composition according to claim 9, wherein the pharmaceutical composition further comprises a second therapeutic agent present in an amount effective to treat AML.

13. The pharmaceutical composition according to claim 12, wherein the amount of the second therapeutic agent is a suboptimal dose.

14. The pharmaceutical composition according to claim 13, wherein the amount of the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof is a suboptimal dose.

15. The pharmaceutical composition according to claim 12, wherein the second therapeutic agent is a chemotherapeutic agent that inhibits DNA synthesis, topoisomerase, FLT3, IDH1, or Syk.

16. The pharmaceutical composition according to claim 12, wherein the second therapeutic agent is daunorubicin; idarubicin; midostaurin; cytarabine; histamine dihydrochloride, alone or in combination with interleukin 2; daunorubicin in combination with cytarabine; gemtuzumab ozogamicin; enasidenib; ivosidenib; TAK-659; entospletinib; quizartinib; gilteritinib; venetoclax; fludarabine; azacitidine; topotecan; arsenic trioxide; cerubidine; cyclophosphamide; daunorubicin hydrochloride; glasdegib maleate; dexamethasone; doxorubicin hydrochloride; enasidenib mesylate; gilteritinib fumarate; idarubicin hydrochloride; mitoxantrone hydrochloride; thioguanine; vincristine sulfate; or any combination thereof.

17. The pharmaceutical composition according to claim 16, wherein the second therapeutic agent is quizartinib.

18. A kit, comprising:
6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or a phosphate-containing prodrug thereof; and
instructions for treating AML with the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof.

19. The kit according to claim 18, wherein the phosphate-containing prodrug is fostamatinib, fostamatinib disodium hexahydrate, sodium (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl phosphate, or a combination thereof.

20. The kit according to claim 18, where the amount of 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof, in the kit is a suboptimal dose.

21. The kit according to claim 18, further comprising a second therapeutic agent for treating AML.

22. The kit according to claim 21, where the second therapeutic agent is present at a suboptimal dose.

23. The kit according to claim 22, where the amount of the 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof in the kit is a suboptimal dose.

24. The kit according to claim 21, wherein the second therapeutic agent is a chemotherapeutic agent that inhibits DNA synthesis, topoisomerase, FLT3, IDH1, or Syk.

25. The kit according to claim 21, wherein the second therapeutic agent is daunorubicin; idarubicin; midostaurin; cytarabine; histamine dihydrochloride, alone or in combination with interleukin 2; daunorubicin in combination with cytarabine; gemtuzumab ozogamicin; enasidenib; ivosidenib; TAK-659; entospletinib; quizartinib; gilteritinib; venetoclax; fludarabine; azacitidine; topotecan; arsenic trioxide; cerubidine; cyclophosphamide; daunorubicin hydrochloride; glasdegib maleate; dexamethasone; doxorubicin hydrochloride; enasidenib mesylate; gilteritinib fumarate; idarubicin hydrochloride; mitoxantrone hydrochloride; thioguanine; vincristine sulfate; or any combination thereof.

26. The kit according to claim 21, wherein the second therapeutic agent is quizartinib.

27. A method, comprising:
identifying a subject that has, or is at risk of developing, acute myeloid leukemia (AML); and
treating the subject with a pharmaceutical composition comprising (i) 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one, or the phosphate-containing prodrug thereof, and (ii) a second therapeutic agent.

28. The method of claim 27, wherein the second therapeutic agent is selected from daunorubicin; idarubicin; midostaurin; cytarabine; histamine dihydrochloride, alone or in combination with interleukin 2; daunorubicin in combination with cytarabine; gemtuzumab ozogamicin; enasidenib; ivosidenib; TAK-659; entospletinib; quizartinib; gilteritinib; venetoclax; fludarabine; azacitidine; topotecan; arsenic trioxide; cerubidine; cyclophosphamide; daunorubicin hydrochloride; glasdegib maleate; dexamethasone; doxorubicin hydrochloride; enasidenib mesylate; gilteritinib fumarate; idarubicin hydrochloride; mitoxantrone hydrochloride; thioguanine; vincristine sulfate; or any combination thereof.

29. The method according to claim 27, wherein identifying a subject that has, or is at risk of developing, acute myeloid leukemia (AML) comprises identifying a subject having an FLT3-ITD mutation.

30. The method according to claim 27, wherein the second therapeutic agent is an FLT3 inhibitor.

31. The method of claim 27, wherein identifying a subject that has, or is at risk of developing, acute myeloid leukemia (AML) comprises identifying a subject having high Syk activity.
